(19) United States
(12) Patent Application Publication
(10) Pub. No.: US 2015/0367122 A1
(43) Pub. Date: Dec. 24, 2015

(54) PATTERNED CARBON NANOTUBE ELECTRODE

(71) Applicant: The University of Memphis, Memphis, TN (US)

(72) Inventors: Bashir I. Morshed, Germantown, TN (US); Md N. Sahadat, Memphis, TN (US); Sergi Consul-pacareu, Memphis, TN (US)

(21) Appl. No.: 14/725,885

(22) Filed: May 29, 2015

Related U.S. Application Data
(60) Provisional application No. 62/005,390, filed on May 30, 2014.

(51) Int. Cl. A61N 1/04; A61B 5/04

(52) U.S. Cl. CPC A61N 1/0488; A61B 5/04; A61B 2562/0209; A61B 2562/0214

(57) ABSTRACT

The present disclosure provides an electrode, including a substrate and a plurality of carbon nanotube pillars disposed on the substrate, wherein at least two of the carbon nanotube pillars are disposed at a predetermined distance from each other. The invention further provides neurological and physiological sensing and stimulation techniques that utilize the electrode of the inventions, and to obtain data thereof for short or long duration using the electrodes of the invention.

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

### CROSS REFERENCE TO RELATED APPLICATION

The present application claims priority to U.S. Provisional Application No. 62/005,390, filed May 30, 2014, the entire contents of which is incorporated herein for all purposes by this reference.

### FIELD OF THE DISCLOSURE

The present disclosure relates to electrodes and physiological sensors. More particularly, the present disclosure relates to medical electrodes comprising a patterned carbon nanotube array for use in dry physiological sensor devices.

### DESCRIPTION OF THE RELATED ART

Many physiological sensing of human body requires conductive or impedimetric electrical sensing of biopotentials or biological resistances for diagnostics, monitoring and therapy. Examples of such physiological data are EEG (electroencephalography), ECG or EKG (electrocardiography), EMG (Electromyography), and GSR (Galvanic Skin Response). Traditional sensors are wetted (e.g., with saline solution) or include a gel interface with the electrode material (e.g., Ag/AgCl). Traditional interfacing of conductive fluid or gel only work for a short duration, as the performance of the sensors deteriorates with time primarily due to evaporation of the conductive fluid or gel.

Dry electrodes are thought to be more suitable for long duration sensing, as well as providing ease of use. Some dry electrode technologies have been disclosed elsewhere (e.g., conductive polymer, PDMS, or metallic pin electrodes), but each suffers from various problems, including poor conductivity, oxidation over time, skin breathing, and high interfacial noise.

Many physiological sensing devices operate at the skin interface. The epidermis, the outermost layer of the skin, contains two major layers: stratum corneum (SC) and stratum germinativum (SG). The SC has electrical isolation characteristics as it consists of dead cells, while the SG is electrically conductive as it is composed of living cells. The next layer is the dermis, which contains nerve endings, blood vessels, and oil glands.

Traditionally, devices that operate at the skin are based on conventional wet or gel-based electrodes, which use a wet solution or gel to maintain an impedance path between the skin and the electrode. However, the gradual decrease of conductivity of electrolytic gel due to drying leads to degrading signal quality.

For long duration sensing, dry electrodes are thought to provide superior performance. However, dry metallic plate electrodes can suffer from oxidation, large half-cell potential drop at the metal-tissue interface, and inability to maintain contact through rough skin surfaces or in the presence of hairs. Electrodes with metallic pins additionally impose threat of injury by puncturing skin. Some other competitive dry electrode technology include conductive polymer and PDMS, all of which have high impedance and low contact area with skin surface in the presence of hairs that tend to degrade signal quality. Previous attempts to create suitable carbon nanotube-based electrodes as a solution to the problems associated with dry-type electrodes have not been successful primarily due to the fact that the nanotubes were not pattered, and very small height of the nanotubes (tens of micrometer range).

Given the lack of available efficacious dry-type electrodes in the art, there is a need for new types of dry-type electrodes, and in particular, carbon nanotube-based dry electrodes for use in physiological sensing instruments and methods.

The above information disclosed in this Background section is only for enhancement of understanding of the background of the disclosure and therefore it may contain information that does not form the prior art that is already known in this country to a person of ordinary skill in the art.

### SUMMARY OF THE DISCLOSURE

The presently claimed and disclosed invention relates to a novel dry nanotube-based electrode system for use in physiological sensing instruments and methods that overcomes the problems recognized in the art for dry-based sensors. The electrode system comprises a patterned vertically aligned carbon nanotube (sometimes referred to herein as “pvCNT”) system for physiological signal sensing from human body. The pvCNT electrodes (or otherwise known herein as “sensors”) can maintain good conductivity through any type of skin, e.g., rough skin, and through thin layers of hairs, e.g., hairs of about 1 mm or more. The pvCNT electrodes of the invention advantageously do not show degraded conductivity over time and are capable of capturing signals in vitro with low-noise. In addition, the pvCNT electrodes of the invention can operate for long durations, e.g., for a continuous or discontinuous use up to at least an hour, or at least 2, 10, 24, 36, 72, or more hours, or even days and/or weeks and/or even months of continuous or discontinuous operation. The beneficial and advantageous properties of the electrodes claimed and disclosed herein are in part due to stable properties of the carbon nanotube structures and pattered vertical growth of the carbon nanotubes on the substrate that composes the electrodes of the invention.

Accordingly, in one aspect, the present disclosure provides an electrode, including a substrate and a plurality of carbon nanotube pillars disposed on the substrate, wherein at least two of the carbon nanotube pillars are disposed at a predetermined distance from each other.

In certain embodiments, the substrate can include stainless steel, polymer, metal, composite, copper, tin, or any other suitable material known in the art. In some embodiments, the substrate is about 0.002 inches thick. In other embodiments, the substrate is flexible and/or pliable such that is easier to conform to the shape of the region of the human body against which it may be pressed, e.g., take on the contours or shape of a human subject over a bodily region over which a physiological measurement is obtained.

In still other embodiments, the substrate can have a thickness that is between about 0.001-0.01 mm, or between about 0.005-0.05 mm, or between about 0.01-0.1 mm, or between about 0.05-1.0 mm, or between about 0.1-2.0 mm, or between about 0.5-10.0 mm, or between about 1.0-50.0 mm, or any suitable thickness that that the substrate is capable of sufficiently operating as a substrate for attachment of the carbon nanotube pillars and for conforming effectively to a bodily site of a subject.

In certain embodiments, the substrate comprises a plurality of carbon nanotube pillars affixed thereon, disposed in a patterned array. In some embodiments, the plurality of carbon nanotube pillars can include at least one rectangular pillar. In other embodiments, the plurality of carbon nanotube pillars can include at least one cylindrical pillar. In various other embodiments, the array of carbon nanotube pillars affixed onto the substrate can be formed of individual pillars that of uniform shape, sized, height, and spacing. In other embodiments, the array of carbon nanotube pillars affixed onto the substrate can be formed of individual pillars that are of dissimilar shape, size, height, and/or space, or any combination of those features being uniform or dissimilar.

In certain embodiments, the plurality of carbon nanotube pillars can be about 1 mm in height. In other embodiments, the plurality of carbon nanotube pillars can range in height from about 0.001-0.01 mm, or between about 0.005-0.05 mm, or between about 0.01-0.1 mm, or between about 0.05-1.0 mm, or between about 0.1-2.0 mm, or between about 0.5-10.0 mm, each of uniform or non-uniform size.

The plurality of carbon nanotube pillars can be about 100 μm in width or diameter, or other suitable dimensions. In certain other embodiments, the plurality of carbon nanotube pillars can be about 200 μm in width or diameter. In still other embodiments, the plurality of carbon nanotube pillars can be about 50, 100, 200, 500 μm, or other suitable dimensions in width or diameter.

In still other embodiments, the spacing between the nanotube carbon pillars can approximate the distance between the nanotube carbon pillars.

In some embodiments, the at least two carbon nanotube pillars can be spaced apart by about 50 μm. The at least two carbon nanotube pillars can be spaced apart by about 100 μm. In some embodiments, the at least two carbon nanotube pillars can be spaced apart by about 200 μm. The at least two carbon nanotube pillars can be spaced apart by about 500 μm, or other suitable dimensions.

In some embodiments, the nanotube pillars are arranged or patterned in a two-dimensional array configuration with the similar or dissimilar spacing in x- and y-directions. In other embodiments, the pillars can be arranged or patterned in hexagonal, circular, ring, or any other geometric configurations.

In still other aspect, the present invention relates to the use of the carbon nanotube-based electrodes of the invention in a physiological sensing device, such as, as an EEG (electroencephalography), ECG or EKG (electrocardiography), EMG (Electromyography), and GSR (Galvanic Skin Response).

In yet another aspect, the present invention relates to a physiological sensing device, such as an EEG (electroencephalography), ECG or EKG (electrocardiography), EMG (Electromyography), and GSR (Galvanic Skin Response), that comprises a carbon nanotube-based electrode of the present invention.

In still other aspects, the present invention relates to methods for obtaining and/or measuring physiological data on a subject in need thereof comprising sensing the physiological data with a sensing device, such as an EEG (electroencephalography), ECG or EKG (electrocardiography), EMG (Electromyography), and GSR (Galvanic Skin Response), that comprises a carbon nanotube-based electrode of the present invention. In yet further aspects, the invention provides methods for physiological stimulation, comprising using a carbon nanotube-based electrode of the invention. The invention also provides methods for neurological stimulation, comprising using a carbon nanotube-based electrode of the invention. The invention also provides methods for muscle stimulation, comprising using a carbon nanotube-based electrode of the invention.

In yet another aspect, a physiological stimulation electrode is provided that comprises a carbon nanotube based electrode of the invention. A neurological stimulation electrode is also provided that comprises a carbon nanotube based electrode of the invention. A muscle stimulation electrode is also provided comprising a carbon nanotube based electrode of the invention.

A hybrid sensing and stimulation electrode of the invention is also provided that comprises two or more modalities as disclosed herein, including two or more of stimulation and/or sensing, including two or more of physiological stimulation, neurological stimulation, muscle stimulation, EEG (electroencephalography), ECG or EKG (electrocardiography), EMG (Electromyography), and GSR (Galvanic Skin Response).

Where applicable or not specifically disclaimed, any one of the embodiments described herein are contemplated to be able to combine with any other one or more embodiments, even though the embodiments are described under different aspects of the invention.

These and other embodiments are disclosed or are obvious from and encompassed by, the following Detailed Description.

### BRIEF DESCRIPTION OF THE DRAWINGS

The above and other features of the present disclosure will now be described in detail with reference to certain exemplary embodiments thereof illustrated the accompanying drawings which are given herein below by way of illustration only, and thus are not limitative of the present disclosure, and wherein:

FIG. 1 illustrates a partial, magnified perspective view of an embodiment of a carbon nanotube (CNT) electrode in accordance with the present disclosure.

FIG. 2 is an SEM image of a patterned array of rectangular CNT pillars.

FIGS. 3A and 3B are SEM images of CNT pillars at high magnification, showing aligned CNT fibers on the edges of the CNT pillars.

FIG. 4A shows an image of a CNT electrode having substantially rectangular CNT pillars of about 1 mm in height and about 100 μm wide on each side, shown with about a 50 μm gap between each CNT pillar.

FIG. 4B shows an image of a CNT electrode having substantially rectangular CNT pillars of about 1 mm in height and about 100 μm wide on each side, shown with about a 100 μm gap between each CNT pillar.

FIG. 4C shows an image of a CNT electrode having substantially rectangular CNT pillars of about 1 mm in height and about 100 μm wide on each side, shown with about a 200 μm gap between each CNT pillar.

FIG. 4D shows an image of a CNT electrode having substantially rectangular CNT pillars of about 1 mm in height and about 100 μm wide on each side, shown with about a 500 μm gap between each CNT pillar.

FIG. 5 shows a CNT electrode disposed on a flexible circuit board.

FIG. 6A shows impedance characterization of pvCNT sensors as being compared with a commercial gel electrode (GS-26).

FIG. 6B shows impedance characterization of pvCNT as being compared with a commercial wet electrode (Emotiv Electrode).

FIG. 7 shows the charted results of a long duration study of pvCNT impedance.

FIG. 8 shows half-cell potential experiment results (Legends: pvCNT=CNT, Commercial gel electrode=GS-26, Baseline of applied ECG signal=SG-OSC).

FIG. 9 shows the stimulated signal applied by CNT electrode and measured across the other side of the agar gel phantom model of skin.

FIG. 10A is an image of submerged sensors in physiological solution.

FIG. 10B is an image of a CNT sensor after 24 hours of submersion in physiological solution.

### DETAILED DESCRIPTION OF THE EMBODIMENTS

The present invention relates in part to the surprising finding that carbon nanotube-based electrodes having a patterned array of nanotubes of a characteristic height, among other properties, demonstrated superior properties as an electrode material suitable as a dry sensor for use in a physiological sensor device, such as an EEG (electroencephalography), ECG or EKG (electrocardiography), EMG (Electromyography), or GSR (Galvanic Skin Response). In one embodiment, the electrode of the invention is particularly suited for measuring physiological data through or via the skin and which represents an improvement over prior wet or dry type electrodes of the prior art.

As will be appreciated, the epidermis contains two major layers: the stratum corneum (SC) and stratum germinativum (SG). The SC has electrical isolation characteristics as it consists of dead cells, while the SG is electrically conductive as it is composed of living cells. The next layer is the dermis, which contains nerve endings, blood vessels, and oil glands. Traditional physiological sensors are wet or gel based (Ag/AgCl) type; however, their performance is not optimal. To overcome the electrical isolation property of the SC and to maintain a low-impedance path from the electrode to the skin tissue, conventional wet electrodes uses the wet solution or gel that maintains an impedance path between the skin and the electrode. The gradual decrease of conductivity of electrolytic gel due to drying leads to degrading signal quality. This is particularly problematic for long duration sensing.

To overcome this problem in the art, dry electrode sensing is thought to provide superior performance as there is no wet or gel requirement. However, dry metallic plate electrodes known in the art suffer from oxidation issues, large half-cell potential drops at the metal-tissue interface, and the inability to maintain contact through rough skin surfaces or in the presence of hair. Metallic pin electrodes additionally impose threat of injury by puncturing skin. Currently-known dry electrode technology includes conductive polymers and PDMS, for example; however, all suffer from most of the abovementioned limitations, including high impedance and degraded signal quality. Carbon nanotube based electrode systems have been attempted, for example carbon nanotube grown on substrates or composite with PDMS, but have not been shown to be functionally successful, in part, due the realization by the inventors that (1) carbon nanotubes were not patterned in the earlier sensors of the state of the art, and (2) the height of the carbon nanotubes was very small (e.g., typically in the micron range).

Accordingly, the presently claimed and disclosed invention relates to a novel dry nanotube-based electrode system for use in physiological sensing instruments and methods that overcome the problems recognized in the art for dry-based sensors. The electrode system of the present invention is structurally very different from the state of the art systems. In particular, the electrodes of the invention comprise a patterned vertically aligned carbon nanotube (sometimes referred to herein as “pvCNT”) system for physiological signal sensing from human body. The pvCNT electrodes (or otherwise known herein as “sensors”) can maintain good conductivity through any type of skin, e.g., rough skin, and through hairs, e.g., hairs of about 1 mm or more. The pvCNT electrodes of the invention advantageously do not show degraded conductivity over time and are capable of capturing signals with low-noise. In addition, the pvCNT electrodes of the invention can operate for long durations, e.g., for a continuous or discontinuous use up to at least an hour, or at least 2, 10, 24, 36, 72, or more hours, or even days and/or weeks and/or months of continuous or discontinuous operation. The beneficial and advantageous properties of the electrodes claimed and disclosed herein are in part due to stable properties of the carbon nanotube structures and pattered vertical growth of the carbon nanotubes on the substrate that composes the electrodes of the invention.

Accordingly, in one aspect, the invention in part provides dry electrodes that have the potential for long duration sensing and ease of use. The technology described and claimed herein represents a novel dry physiological sensor that operates for a long duration, is easy to use, breathable, and can penetrate rough skin or hair, among other features and advantages.

Among other aspects and advantages, the dry electrodes of the present invention provides at least the following advantages: (a) dry physiological sensor with low interfacial potential; (b) maintains conductivity over a long time in vitro; (c) ability to maintain connectivity through rough skin surface and hair; (d) flexible substrate that conforms to head contour, thus providing ease of use; and (e) breathable (i.e., can allow skin to breath due to gaps between pvCNT pillars).

In one aspect, the present invention relates to pvCNT electrodes for use in physiological sensing devices, such as, but not limited to EEG (electroencephalography), ECG or EKG (electrocardiography), EMG (Electromyography), or GSR (Galvanic Skin Response).

In certain embodiments, the electrodes of the invention include a substrate and a plurality of carbon nanotube pillars disposed on the substrate, wherein at least two of the carbon nanotube pillars are disposed at a predetermined distance from each other.

The substrate on which the carbon nanotubes are assembled can be any suitable material in the art, including, for example, stainless steel, polymer, metal, composite, copper, tin, or any other suitable material known in the art. The nanotubes may be of any type known in the art, without limitation, for example, single-walled nanotubes, multi-walled nanotubes, torus nanotubes, nanobuds, graphenated carbon nanotubes (g-CNTs), nitrogen-doped carbon nanotubes, peapod nanotubes, cup-stacked carbon nanotubes, as well as other known types of nanotubes.

Any suitable method for fabricating the nanotubes of the invention may be used. For example, the nanotube fabrication methodologies described in the following publications can be used in accordance with the invention, each of which are incorporated by reference in their entireties: U.S. Published Application Nos. 20140052037, entitled, SHEET-LIKE CARBON NANOTUBE-POLYMER COMPOSITE MATERIAL; US 20140045303, entitled, CONTACTS-FIRST SELF-ALIGNED CARBON NANOTUBE TRANSISTOR WITH GATE-ALL-AROUND; US20140044873, entitled, SINGLE-WALLED CARBON NANOTUBE (SWCNT) FABRICATION BY CONTROLLED CHEMICAL VAPOR DEPOSITION (CVD); US20140042490, entitled, NANOTUBE SEMICONDUCTOR DEVICES; US20140042385, entitled, CONTACTS-FIRST SELF-ALIGNED CARBON NANOTUBE TRANSISTOR WITH GATE-ALL-AROUND; US20140041791, entitled APPARATUS FOR GROWING CARBON NANOTUBE FORESTS, AND GENERATING NANOTUBE STRUCTURES THEREFROM, AND METHOD; US20140037938, entitled CARBON NANOTUBE ENABLED HYDROPHOBIC-HYDROPHILIC COMPOSITE INTERFACES AND METHODS OF THEIR FORMATION; US20140037895, entitled COMPOSITE CARBON NANOTUBE STRUCTURE, US20140034906, entitled, CARBON NANOTUBE SEMICONDUCTOR DEVICES AND DETERMINISTIC NANOFABRICATION METHODS; US20140034881, entitled CARBON NANOTUBE-RADICAL POLYMER COMPOSITE AND PRODUCTION METHOD THEREFOR; US20140034633, entitled CARBON NANOTUBE THIN FILM LAMINATE RESISTIVE HEATER; US20140030950, entitled METHOD FOR MAKING CARBON NANOTUBE FIELD EMITTER; US20140030504, entitled ELECTROLESS PLATED FILM INCLUDING PHOSPHORUS, BORON AND CARBON NANOTUBE; US20140030183, entitled CARBON NANOTUBE MANUFACTURING METHOD; US20140028178, entitled CARBON NANOTUBE FIELD EMITTER; US20140027678 entitled METHOD FOR PREPARING CARBON NANOTUBE OR CARBON MICROTUBE; US20140027404, entitled METHOD FOR MAKING CARBON NANOTUBE NEEDLE; US20140026535, entitled HIGH SPECIFIC IMPULSE SUPERFLUID AND NANOTUBE PROPULSION DEVICE, SYSTEM AND PROPULSION METHOD; US20140023588, entitled METHOD OF DRUG DELIVERY BY CARBON NANOTUBE CHITOSAN NANOCOMPLEXES; US20140023116, entitled CARBON NANOTUBE TEMPERATURE AND PRESSURE SENSORS; and US20140021403, entitled CARBON NANOTUBE COMPOSITE AND METHOD OF MANUFACTURING THE SAME.

The patterning is performed with a mask that confines catalyst deposition. Typical catalyst deposition processing for vertically aligned carbon nanotube growth includes the deposition of alumina and iron layers (e.g. 10 nm of Al2O3 followed by 1 to 2 nm of iron) by sputtering process. For patterning, the photoresist is imaged and developed prior to catalyst deposition. After deposition, the resist is stripped and the patterned substrates are diced to the size of the sensor (e.g. 10 mm dia disc). Laser cutting tools can be used for such dicing. The carbon nanotubes are then grown by following several steps: heat-up, anneal, growth, and cool-down. Heat-up process breaks catalyst film to islands. Fast heat-up and short annealing nucleates smaller diameter nanotubes, while longer anneals lead to larger diameter nanotubes. Rapid annealing at high temperature (e.g. 500 C) allows fast growth of carbon nanotubes.

The carbon nanotube pillars of the invention may be fabricated by any known and/or suitable technical means or methodology, including for example, the arc discharge method, laser ablation method, plasma torch method, thermal growth, and chemical vapor deposition method, as well as other suitable methods.

In certain aspects, the carbon nanotube pillars of the invention may be used as sensor components of a physiological sensing device, such as an EEG (electroencephalography), ECG or EKG (electrocardiography), EMG (Electromyography), or GSR (Galvanic Skin Response). The electrodes of the invention may be used with any known sensing system. For example, the electrodes of the invention may be used with the electrocardiography devices, such as those described in US20130331721, entitled ELECTROCARDIOGRAPH SYSTEM; US20130331720, entitled ELECTROCARDIOGRAPH SYSTEM; US20120143020, entitled EEG KIT; US20110270048, entitled SYSTEMS AND METHODS FOR PPG SENSORS INCORPORATING EKG SENSORS; US20110245690, entitled SYSTEMS AND METHODS FOR MEASURING ELECTROMECHANICAL DELAY OF THE HEART, each of which are incorporated by reference. In addition, the electrodes of the invention may be used with the electroencephalography devices described in US20130172721, entitle DEVICE AND METHOD FOR PERFORMING ELECTROENCEPHALOGRAPY and US20130096440, entitled PORTABLE FETAL EEG-RECORDING DEVICE AND METHOD OF USE, each of which are incorporated by reference. Moreover, the electrodes of the invention may be used with the electromyography devices described in US20120188158, entitled WEARABLE ELECTROMYOGRAPHY-BASED HUMAN-COMPUTER INTERFACE; US20120184838, entitled NON-INVASIVE DEEP MUSCLE ELECTROMYOGRAPHY; US20120172682, entitled METHOD AND APPARATUS FOR BIOMETRIC ANALYSIS USING EEG AND EMG SIGNALS; and US20120137795, entitled RATING A PHYSICAL CAPABILITY BY MOTION ANALYSIS, each of which are incorporated by reference.

The spacing between the carbon nanotube pillars can be any suitable space, and can include regular or irregular spacing patterns. In some embodiments, the at least two carbon nanotube pillars can be spaced apart by about 50 μm. The at least two carbon nanotube pillars can be spaced apart by about 100 μm. In some embodiments, the at least two carbon nanotube pillars can be spaced apart by about 200 μm. The at least two carbon nanotube pillars can be spaced apart by about 500 μm, or other suitable dimensions.

In still other embodiments, the spacing between the nanotube carbon pillars can approximate the distance between the nanotube carbon pillars.

The height of the pillars can be any suitable height, wherein the height among individual pillars can be regular or irregular. In certain embodiments, the plurality of carbon nanotube pillars can be about 1 mm in height. In other embodiments, the plurality of carbon nanotube pillars can ranged in height from about 0.001-0.01 mm, or between about 0.005-0.05 mm, or between about 0.01-0.1 mm, or between about 0.05-1.0 mm, or between about 0.1-2.0 mm, or between about 0.5-10.0 mm, each of uniform or non-uniform size.

The plurality of carbon nanotube pillars can be about 100 μm in width or diameter. In certain other embodiments, the plurality of carbon nanotube pillars can be about 200 μm in width or diameter. In still other embodiments, the plurality of carbon nanotube pillars can be about 50, 100, 200, 500 μm, or other suitable dimensions in width or diameter.

Hereinafter, exemplary embodiments of the present disclosure will be described in detail, referring to the accompanying drawings.

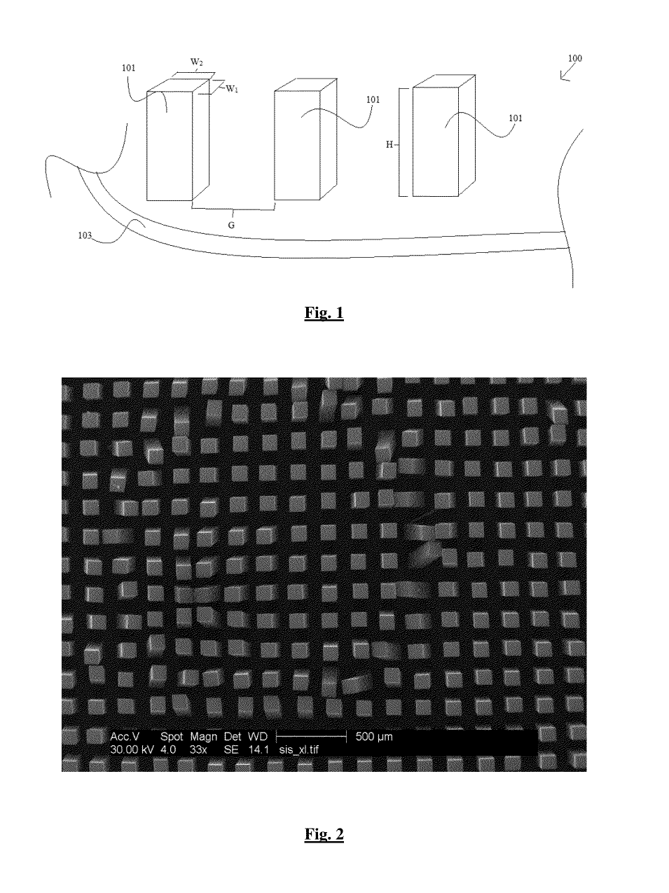

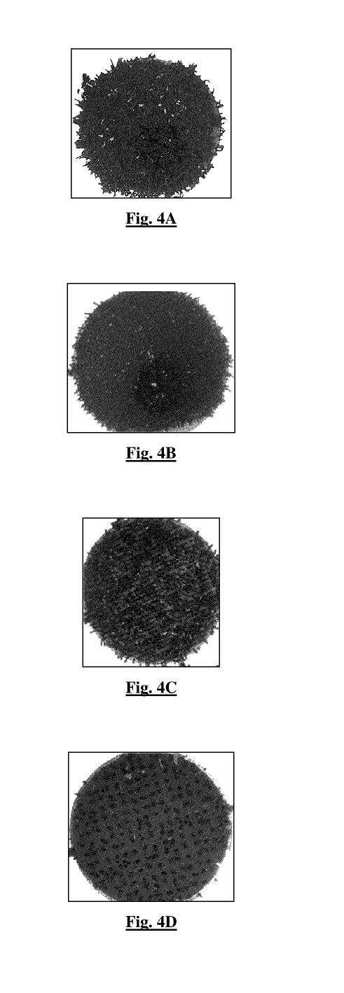

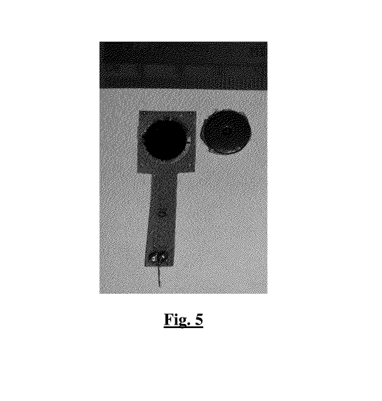

Referring to FIGS. 1-5, the present disclosure provides an electrode 100 including a substrate 103 and a plurality of carbon nanotube (CNT) pillars 101 disposed on the substrate 103, wherein at least two of the CNT pillars 101 are disposed at a predetermined distance “G” from each other. The substrate 103 can include stainless steel or any other suitable material. The substrate 103 can also include any suitable dimensions, size, shape (e.g., circular wafer shape), thickness, and/or the like. In some embodiments, the substrate 103 is about 0.002 inches thick and about 10 mm in diameter.

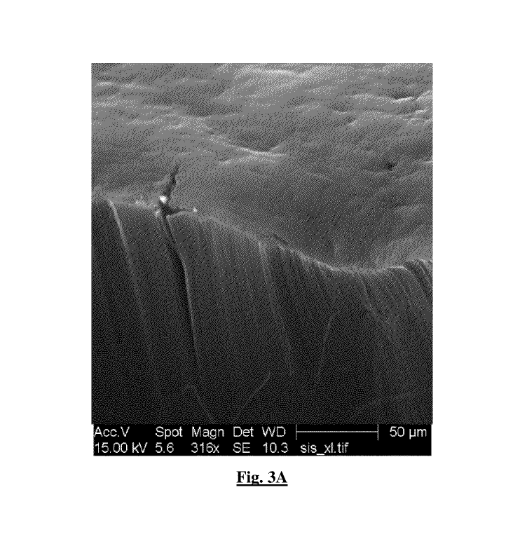

The plurality of CNT pillars 101 can be disposed in a patterned array (e.g., one or more repeating patterns) as shown in FIG. 2, or in any other suitable manner.

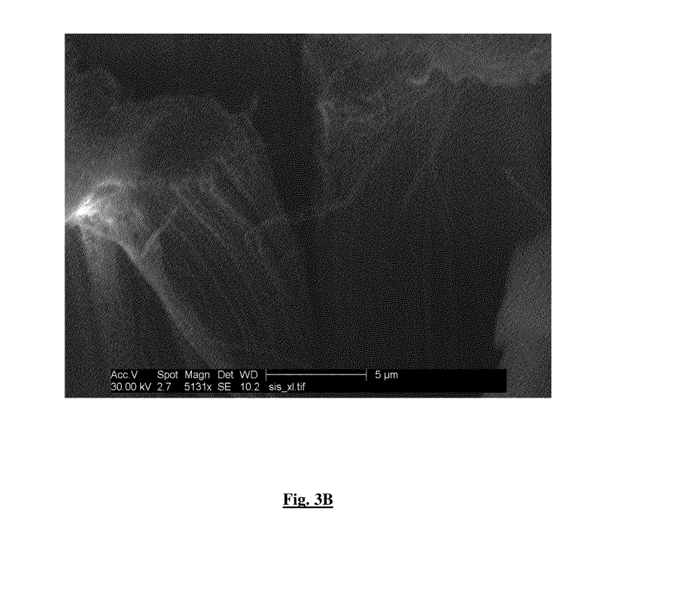

The CNT pillars 101 can be comprised of a plurality of CNT fibers or any other suitable material. In some embodiments the CNT fibers are aligned vertically (e.g., as shown in FIGS. 3A and 3B). In some embodiments, the plurality of CNT pillars 101 can include at least one rectangular shaped pillar such as those shown in FIGS. 1-5. However, it is contemplated that the plurality of CNT pillars 101 can include, alternatively or conjunctively, any other shaped CNT pillar (e.g., at least one cylindrical pillar).

The plurality of CNT pillars 101 can be any suitable height “H”, e.g., about 1 mm. The plurality of CNT pillars 101 can also include any suitable width, diameter, or cross-sectional area. For example, for a rectangular CNT pillar 101 as shown, a first width W₁ can be about 100 μm and a second width W₂ can be about 100 μm to form a substantially square cross-section. A diameter of 100 μm is contemplated for circular embodiments. Irregular shaped CNT pillars 101 can be designed to include a predetermined cross-sectional area (e.g., about 10,000 μm²) instead of using difficult geometric reference dimensions to define size.

The predetermined distance “G” that separates at least two CNT pillars 101 can be any suitable distance (e.g., about 10 μm to about 1000 μm). In some embodiments, the at least two CNT pillars 101 can be spaced apart by about 50 μm. In other embodiments, the at least two CNT pillars 101 can be spaced apart by about 100 μm. In some embodiments, the at least two CNT pillars 101 can be spaced apart by about 200 μm. In another embodiment, the at least two CNT pillars 101 can be spaced apart by about 500 μm. Any other suitable ranges and combinations of the above are contemplated on a single electrode 100.

In at least one aspect of this disclosure, a method includes disposing at least two CNT pillars 101 as disclosed herein on a substrate 103 at a predetermined distance “G” from each other. The method can further include disposing the CNT pillars about 50 μm to about 500 μm apart from each other. The method can also include vertically aligning a plurality of carbon nanotubes to form the CNT pillars 101. The CNT pillars 101 can be formed on the substrate 103 in any suitable manner known in the art.

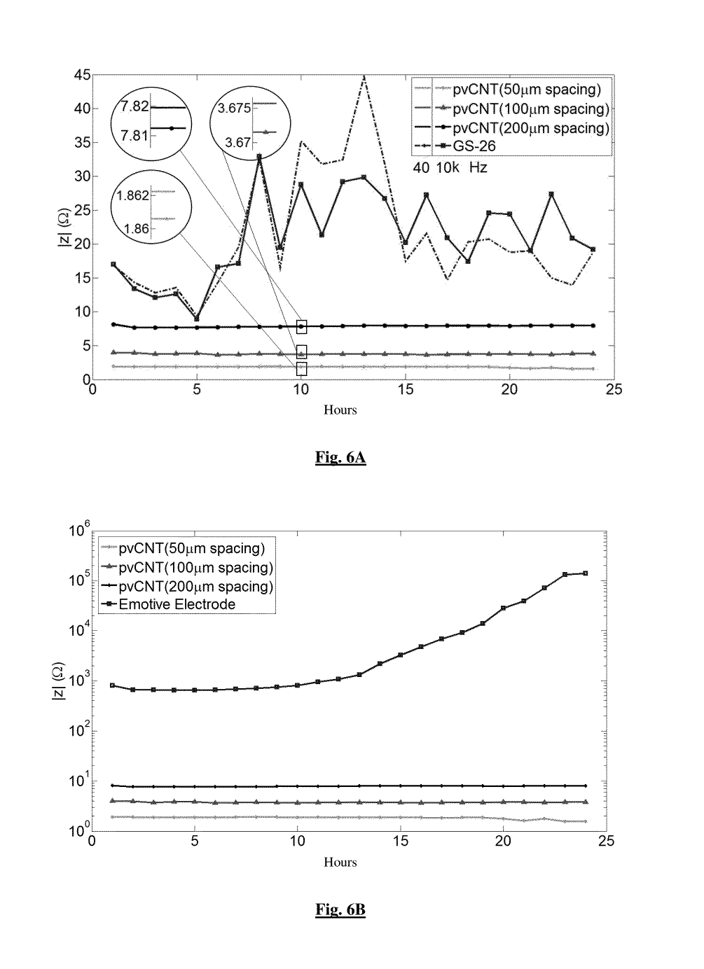

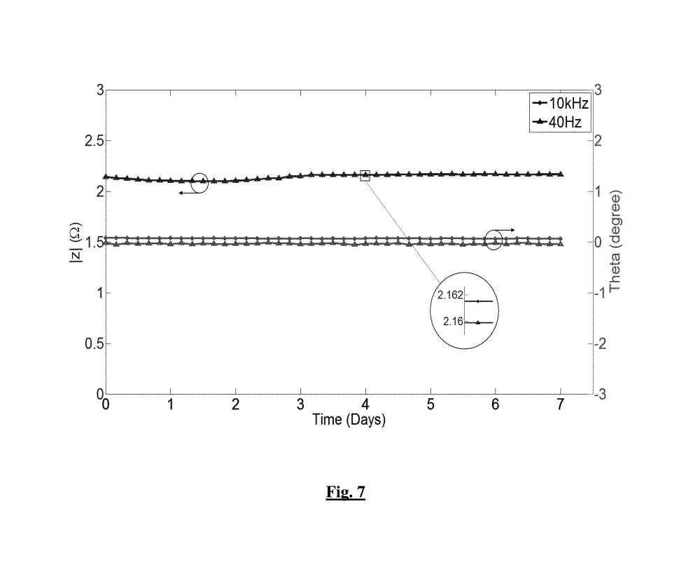

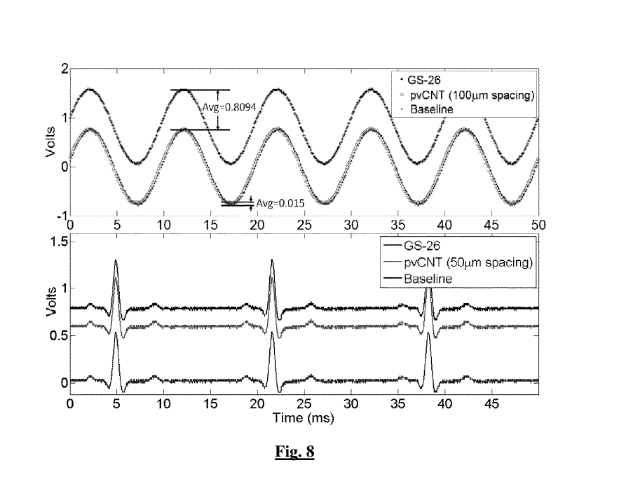

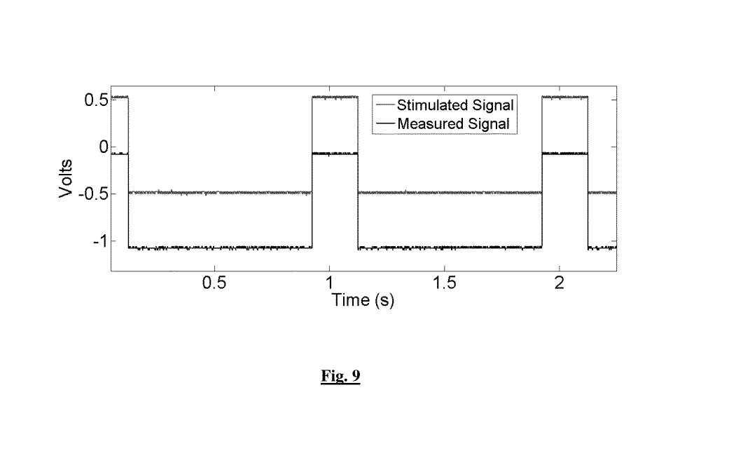

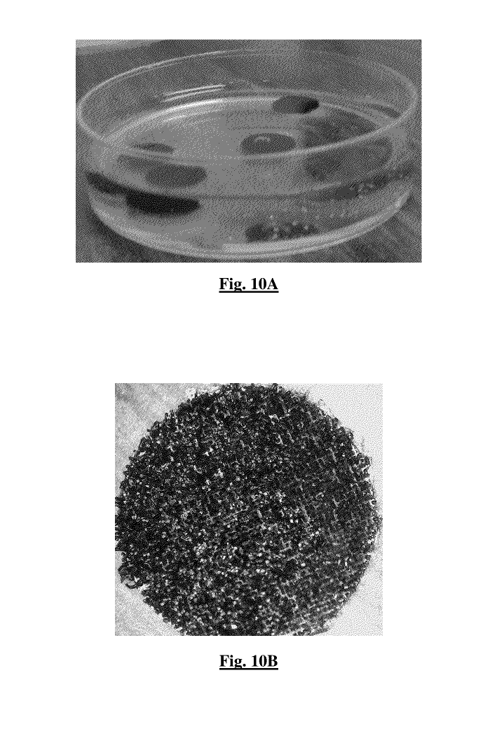

The electrode 100 having CNT pillars 101 as disclosed herein can allow physiological signal monitoring through thin layers of hair Carbon nanotube based wearable sensors and stimulators were demonstrated and tested as shown in FIGS. 6A-10B. The pvCNT electrode was also tested for weeklong experiments to show that the degradation of the impedance is minimal. In one embodiment, experimental results, for instance, showed that the potential drops of the electrodes interfaced with Al foil are 809.4 mV and 15 mV for gel electrode and pvCNT electrode, respectively. For both sensing and stimulation experiments, it was found that there were insignificant structural changes of the pvCNT pillar formation. However, it is noted that high axial compression force can cause disintegration of pvCNT pillar formation, which can be improved with various approaches, for example thin film coating.

A dry electrode 100 with a predetermined distance between CNT pillars 101 (e.g., sparsely distributed vertically aligned CNT pillars) can provide improved signal quality (e.g., due to high surface contact area and/or low interfacial potential), an improved ability for long duration of operation (because the electrodes 100 are dry and may not oxidize), conformability (as the electrode 100 can be disposed on a flexible printed circuit board), and breathability (e.g., due to gaps between CNT pillars 101).

Due to the structure of pvCNT pillars with spacing that allows airflow, pvCNT electrode has better breathability. The bristle-like arrangement of the pvCNT electrode will also promote good contact over rough skin surfaces and pores. As small concentration of carbon nanotubes are shown to be not toxic on the epidermal tissue and due to low possibility of skin puncture, the pvCNT electrode can possibly be safer to use.

While this disclosure has been described in connection with what is presently considered to be practical exemplary embodiments, it is to be understood that the disclosure is not limited to the disclosed embodiments, but, on the contrary, is intended to cover various modifications and equivalent arrangements included within the spirit and scope of the appended claims.

1. An electrode, comprising:
a substrate; and
a plurality of carbon nanotube pillars disposed on the substrate, wherein at least two of the carbon nanotube pillars are disposed at a predetermined distance from each other.

2. The electrode of claim 1, wherein the substrate includes stainless steel.

3. The electrode of claim 1, wherein the substrate is about 0.002 inches thick.

4. The electrode of claim 1, wherein the plurality of carbon nanotube pillars are disposed in a patterned array.

5. The electrode of claim 1, wherein the plurality of carbon nanotube pillars include at least one rectangular pillar or at least one cylindrical pillar.

6. (canceled)

7. The electrode of claim 1, wherein the plurality of carbon nanotube pillars are about 1 mm in height.

8. The electrode of claim 1, wherein the plurality of carbon nanotube pillars are about 100 μm in width or diameter.

9. The electrode of claim 1, wherein the at least two carbon nanotube pillars are spaced apart by about 50 μm.

10. The electrode of claim 1, wherein the at least two carbon nanotube pillars are spaced apart by about 100 μm.

11. The electrode of claim 1, wherein the at least two carbon nanotube pillars are spaced apart by about 200 μm.

12. The electrode of claim 1, wherein the at least two carbon nanotube pillars are spaced apart by about 500 μm.

13. A method of obtaining physiological data of a subject comprising measuring a impedance or biopotential of the skin of a subject using a physiological sensing device comprising an electrode through resistive or capacitive mechanism, wherein the electrode comprises a substrate and a plurality of carbon nanotube pillars disposed on the substrate, wherein at least two of the carbon nanotube pillars are disposed at a predetermined distance from each other.

14. The method of claim 13, wherein the substrate includes stainless steel, polymer or other conductive or non-conductive layers.

15. The method of claim 13, wherein the substrate is about 0.002 inches thick.

16. The method of claim 13, wherein the plurality of carbon nanotube pillars are disposed in a patterned array.

17. The method of claim 13, wherein the plurality of carbon nanotube pillars include at least one rectangular pillar or at least one cylindrical pillar.

18. (canceled)

19. The method of claim 13, wherein the plurality of carbon nanotube pillars are about 1 mm in height.

20. The method of claim 13, wherein the plurality of carbon nanotube pillars are about 100 μm in width or diameter.

21. The method of claim 13, wherein the at least two carbon nanotube pillars are spaced apart by about 50 μm.

22. The method of claim 13, wherein the at least two carbon nanotube pillars are spaced apart by about 100 μm.

23. The method of claim 13, wherein the at least two carbon nanotube pillars are spaced apart by about 200 μm.

24. The method of claim 13, wherein the at least two carbon nanotube pillars are spaced apart by about 500 μm.

25. The method of claim 13, wherein the physiological sensing device is an EEG (electroencephalography), ECG or EKG (electrocardiography), EMG (Electromyography), and GSR (Galvanic Skin Response).

26. A physiological sensing device comprising a carbon nanotube-based electrode of claim 1.

27. The physiological sensing device of claim 26, wherein the device is an EEG (electroencephalography), ECG or EKG (electrocardiography), EMG (Electromyography), or GSR (Galvanic Skin Response).

28. A physiological stimulation electrode comprising a carbon nanotube based electrode of claim 1, or a carbon nanotube based electrode of claim 1, or a carbon nanotube based electrode of claim 1.

29.-30. (canceled)

31. A hybrid sensing and stimulation electrode comprising two or more modalities as recited in claim 25.

32. A hybrid sensing and stimulation electrode comprising two or more modalities as recited in claim 26.
